(19) United States
(12) Patent Application Publication
(10) Pub. No.: US 2024/0139287 A1
(43) Pub. Date: May 2, 2024

(54) THERAPEUTIC USES OF DULAGLUTIDE

(71) Applicant: Eli Lilly and Company, Indianapolis, IN (US)

(72) Inventors: Jeffrey S. RIESMEYER, Indianapolis, IN (US); David Bradley WOODWARD, Fishers, IN (US)

(21) Appl. No.: 18/409,277

(22) Filed: Jan. 10, 2024

Related U.S. Application Data
(63) Continuation of application No. 17/599,621, filed on Sep. 29, 2021, now Pat. No. 11,890,325.
(60) Provisional application No. 62/829,717, filed on Apr. 5, 2019.

(51) Int. Cl. A61K 38/26; A61K 31/616; A61K 45/06; A61P 3/10

(52) U.S. Cl. CPC A61K 38/26; A61K 31/616; A61K 45/06; A61P 3/10

(57) ABSTRACT

The present invention relates to methods for reducing the risk of major adverse cardiovascular events in type 2 diabetes mellitus (T2DM) patients with multiple cardiovascular risk factors without established cardiovascular disease or with established cardiovascular disease comprising administering the glucagon like peptide-1 (GLP-1) receptor agonist dulaglutide.

The present invention relates to the field of medicine. More particularly, the present invention relates to methods for reducing the risk of major adverse cardiovascular events in type 2 diabetes mellitus (T2DM) patients with multiple cardiovascular risk factors or established cardiovascular disease comprising administering the glucagon like peptide-1 (GLP-1) receptor agonist dulaglutide.

Patients with T2DM frequently suffer from a variety of comorbidities, one of which is cardiovascular disease (CVD). The incidence of CND in T2DM patients is approximately twice than that in non-diabetic individuals, and modification of CVD risk factors, including diet and exercise, is a standard component of T2DM treatment plans, but CVD-related death remains the most common cause of death in T2DM patients.

The effects of both glucose lowering and non-glucose lowering therapies on the incidence of cardiovascular events have been studied. Studies have shown non-glucose lowering therapies, including statins, such as atorvastatin, renin angiotensin system modulators, such as ramipril and telmisartan, and combinations of perindopril, an angiotensin converting enzyme (ACE) inhibitor and indapamide, a thiazide diuretic (TLD), are capable of reducing the incidence of cardiovascular events in T2DM patients.

Studies on the effects of glucose-lowering therapies on the incidence of cardiovascular events have generated varying results. For example, pioglitazone had a mixed effect on cardiovascular outcomes, basal insulin and dipeptidyl peptidase-4 (DPP-4) inhibitors had a neutral effect on cardiovascular outcomes, and empagliflozin, a sodium-glucose co-transporter-2 (SGLT2) inhibitor, reduced cardiovascular mortality and hospitalization for heart failure.

Similarly, studies on the effects of different agents within the class of GLP-1 receptor agonists on the incidence of cardiovascular events have also generated varying results. On the one hand, lixisenatide was found to not significantly alter the rate of major adverse cardiovascular events or other serious adverse events in patients with established CVD (Pfeffer M A, et al. Lixisenatide in patients with type 2 diabetes and acute coronary syndrome, 373 N. ENGL. J MED. 2247-2257 (2015) (“ELIXA”)), and once weekly exenatide was found to not result in a significant difference in the incidence of major adverse cardiovascular events compared to placebo in a patient population including patients with and without established CVD (Holman R R, et al., Effects of Once-Weekly Evenatide on Cardiovascular Outcomes in Type 2 Diabetes, 377 N. ENGL. J. MED. 1228-1239 (2017) (“EXCSEL”)). On the other hand, albiglutide, liraglutide and semaglutide were found to reduce the risk of major adverse cardiovascular events in patient populations comprised entirely (albiglutide) or predominantly (liraglutide and semaglutide) of patients with established CVD. (Hernandez A F, et al., Albiglutide and Cardiovascular Outcomes in Patients with Type 2 Diabetes and Cardiovascular Disease (Harmony Outcomes): a Double-blind, Randomized Placebo-controlled Trial, 392 LANCET 1519-1529 (2018) (“Harmony Outcomes”); Marso S P, et al., Liraglutide and Cardiovascular Outcomes in Patients with Type 2 Diabetes, 375 N. ENGL. J. MED. 311-322 (2016) (“LEADER”)); Marso S P, et al., Semaglutide and Cardiovascular Outcomes in Patients with Type 2 Diabetes 375 N. ENGL. J. MED. 1834-1844 (2016) (“SUSTAIN-6”)).

Despite the therapies described above, the need for new treatment options capable of reducing the risk of major adverse cardiovascular events in T2DM patients remains. In particular, the need remains for treatment options capable of reducing the risk of major adverse cardiovascular events in T2DM patients who do not already have established cardiovascular disease.

The methods of the present invention seek to meet those needs. Indeed, dulaglutide was recently found to be capable of statistically significantly reducing the risk of major adverse cardiovascular events in a population that included patients both with and without established cardiovascular disease. Moreover, the overall reduction in risk seen was driven by and similar in both patients with and without established cardiovascular disease.

Accordingly, the present invention provides a method of reducing the risk of major adverse cardiovascular events in a patient with type 2 diabetes mellitus, comprising administering dulaglutide in a therapeutically effective amount to the patient once weekly, wherein the patient has type 2 diabetes mellitus and either: multiple cardiovascular risk factors without established cardiovascular disease; or established cardiovascular disease.

In another aspect, the present invention provides a method of reducing the risk of major adverse cardiovascular events in a patient with type 2 diabetes mellitus, comprising: identifying a patient having type 2 diabetes mellitus and either multiple cardiovascular risk factors without established cardiovascular disease or established cardiovascular disease; and administering dulaglutide in a therapeutically effective amount to the patient once weekly.

In another aspect, the present invention provides a method of delaying the occurrence of major adverse cardiovascular events in a patient with type 2 diabetes mellitus, comprising administering dulaglutide in a therapeutically effective amount to the patient once weekly, wherein the patient has type 2 diabetes mellitus and either: multiple cardiovascular risk factors without established cardiovascular disease; or established cardiovascular disease.

In another aspect, the present invention provides a method of improving glycemic control and reducing the risk of first occurrence of a major adverse cardiovascular event in a patient with type 2 diabetes mellitus, comprising administering dulaglutide in a therapeutically effective amount to the patient once weekly, wherein the patient has type 2 diabetes mellitus and either: multiple cardiovascular risk factors without established cardiovascular disease; or established cardiovascular disease.

In another aspect, the present invention provides dulaglutide for use in reducing the risk of major adverse cardiovascular events in a patient with type 2 diabetes mellitus and either: multiple cardiovascular risk factors without established cardiovascular disease; or established cardiovascular disease.

In another aspect, the present invention provides use of dulaglutide for the preparation of a medicament for reducing the risk of major adverse cardiovascular events in a patient with type 2 diabetes and either: multiple cardiovascular risk factors without established cardiovascular disease; or established cardiovascular disease.

Dulaglutide is a human GLP-1 receptor agonist which comprises a dimer of a GLP-1 analog fused at its C-terminus via a peptide linker to the N-terminus of an analog of an Fc portion of an immunoglobulin, and is identified by CAS registry number 923950-08-7, which provides the following chemical name: 7-37-Glucagon-like peptide I [8-glycine,22-glutamic acid,36-glycine] (synthetic human) fusion protein with peptide (synthetic 16-amino acid linker) fusion protein with immunoglobulin G4 (synthetic human Fc fragment), dimer. Each monomer of dulaglutide has the amino acid sequence set forth in SEQ ID NO:1:

(SEQ ID NO: 1)
HGEGTFTSDVSSYLEEQAAKEFIAWLVKGGGGGGGSGGGGSGGGGSAESK
YGPPCPPCPAPEAAGGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLG.

The two monomers are attached by disulfide bonds between the cysteine residues at positions 55 and 58 of SEQ ID NO:1 to form the dimer. Dulaglutide's structure, function, production and use in treating T2DM is described in more detail in U.S. Pat. No. 7,452,966 and U.S. Patent Application Publication No. US20100196405. When used herein, the term “dulaglutide” refers to any GLP-1 receptor agonist protein dimer of two monomers having the amino acid sequence of SEQ ID NO:1, including any protein that is the subject of a regulatory submission seeking approval of a GLP-1 receptor agonist product which relies in whole or part upon data submitted to a regulatory agency by Eli Lilly and Company relating to dulaglutide, regardless of whether the party seeking approval of said protein actually identifies the protein as dulaglutide or uses some other term. Dulaglutide agonizes the GLP-1 receptor resulting in stimulation of insulin synthesis and secretion, and has been shown to provide improved glycemic control in T2DM patients.

It has now been discovered that dulaglutide is capable of reducing the risk of major adverse cardiovascular events in patients having T2DM and with either established cardiovascular disease or without established cardiovascular disease with multiple cardiovascular risk factors.

As noted above, the effects of several other GLP-1 receptor agonists on cardiovascular outcomes (referred to as a cardiovascular outcome trial, or “CVOT”) had been previously studied, and the results of those studies were mixed, with some demonstrating a benefit in patients having T2DM and some not demonstrating such a benefit. Summaries of the enrollment in and composite MACE 3 results from those studies are provided below in Table 1.

TABLE 1
GLP-1 receptor agonist CVOTs enrollment and MACE3 results.

| | | | | | Albiglutide |
|---|---|---|---|---|---|
| | | | | Exenatide | QW |
| | Semaglutide | Liraglutide | Lixisenatide | QW | (Harmony |
| | (SUSTAIN-6) | (LEADER) | (ELIXA) | (EXSCEL) | Outcomes) |
| # of | 3297 | 9340 | 6068 | 14752 | 9463 |
| Patients | | | | | |
| Age | 65 (mean) | 64 (mean) | 60 (mean) | 62 (median) | 64 (mean) |
| Prior | 83%^{a} | 81%^{b} | 100%^{c} | 73%^{d} | 100%^{e} |
| CVD | | | | | |
| Statin Use | 73% | 72% | 93% | 74% | 84% |
| BMI | 33 (mean) | 33 (mean) | 30 (mean) | 32 (median) | 32 (mean) |
| HbA1c | 8.7% (mean) | 8.7% (mean) | 7.7% (mean) | 8.0% (median) | 8.7% (mean) |
| # of | 254 | 1302 | 805 | 1744 | 766 |
| events | | | | | |
| Median | 2.1 years | 3.8 years | 2.1 years | 3.2 years | 1.6 years |
| follow up | | | | | |
| MACE 3 | 0.74 | 0.87 | 1.02 | 0.91 | 0.78 |
| Hazard | (0.58-0.95) | (0.78-0.97) | (0.89-1.17) | (0.83-1.00) | (0.68-0.90) |
| Ratio (CI) | | | | | |

^{a}83.0% established cardiovascular disease including CKD 3+, and 58.8% had established cardiovascular disease without CKD;
^{b}Cardiovascular disease, cerebrovascular disease, PVD, CRF, CHF;
^{c}Acute coronary event within 180 days before screening;
^{d}73% at least one prior cardiovascular event (70% CAD, 24% PAD, 22% cerebrovascular disease);
^{e}Coronary artery disease, cerebrovascular disease or peripheral arterial disease.

As seen in Table 1, the results of three of the studies suggested the agents tested had a positive effect, while the results of two of the studies did not show a statistically significant difference from placebo.

Moreover, even in those studies demonstrating a benefit, the positive results were driven by patients with established cardiovascular disease. As also seen in Table 1, two of the studies included only patients having prior CVD. With respect to the studies that did include patients without established CVD, a comparison of results for patients with established CVD vs. those without established CVD (but with multiple risk factors) in the remaining studies is provided below in Table 2.

TABLE 2
“No Prior CVD” refers to patients without established CVD (but with
multiple risk factors). “Prior CVD” refers to patients with established CVD.

| Agent | Liraglutide | | Semaglutide | | Exenatide QW | |
|---|---|---|---|---|---|---|
| Population | No Prior CVD | Prior CVD | No Prior CVD | Prior CVD | No Prior CVD | Prior CVD |
| | (N = 1742; | (N = 7598; | (N = 562; | (N = 2735; | (N = 3970; | (N = 10782; |
| | 18.7%) | 81.3%) | 17.0%) | 83.0%) | 26.9%) | 73.1%) |
| MACE 3 | 1.20 | 0.83 | 1.00 | 0.72 | 0.99 | 0.90 |
| Hazard | (0.86-1.67) | (0.74-0.93) | (0.41-2.46) | (0.55-0.93) | (0.77-1.28) | (0.82-1.00) |
| ratio (CI) | | | | | | |

| | | |
|---|---|---|
| Interaction P = 0.04 | Interaction P = 0.49 | Interaction P = 0.50 |

As seen above in Table 2, in none of the studies which enrolled patients without established CVD did that population of patients drive improvements in major adverse cardiovascular events. Contrarily, as described in more detail in the Examples below, treatment with dulaglutide was found to be capable of statistically significantly reducing the risk of major adverse cardiovascular events in a population that included patients with and without established cardiovascular disease, and that reduction in risk seen was driven by, and similar in, both of those groups of patients.

When used herein to characterize a patient, the term “established CVD” or “established cardiovascular disease” refers to a patient having one or more of the following: prior myocardial infarction (MI); prior ischemic stroke; prior unstable angina; prior revascularization (coronary, carotid or peripheral); prior hospitalization for ischemia-related events (unstable angina or myocardial ischemia on imaging or need for percutaneous coronary intervention); and prior documented myocardial ischemia.

When used herein, the term “major adverse cardiovascular events” refers to cardiovascular death, non-fatal myocardial infarction and non-fatal stroke. These events are also sometimes referred to as MACE or MACE 3 events. The first to occur of any of these events is a composite endpoint frequently used in CVOTs.

When used herein in relation to major adverse cardiovascular events, the term “risk factors” refers to characteristics of T2DM patients understood to increase their risk for a major adverse cardiovascular event. Such risk factors include in particular any of the following: current tobacco use (any form of tobacco); use of at least 1 approved lipid modifying therapy (e.g., statins such as atorvastatin, rosuvastatin, simvastatin, pravastatin, lovastatin, fluvastatin or pitavastatin; PCSK9 inhibitors, such as evolocumab or alirocumab; and ezetimibe) to treat hypercholesterolemia or a documented untreated low-density lipoprotein cholesterol (LDL-C) ≥3.4 mmol/L (130 mg/dL) within the past 6 months; documented treated or untreated high-density lipoprotein cholesterol (HDL-C) <1.0 mmol/L (40 mg/dL) for men and <1.3 mmol/L (50 mg/dL) for women or triglycerides ≥2.3 mmol/L (200 mg/dL) within the past 6 months; use of at least 1 blood pressure medication to treat hypertension (e.g., angiotensin converting enzyme (ACE) inhibitors, angiotensin receptor blockers (ARBs), thiazidelike diuretics, and dihydropyridine calcium channel blockers) or untreated systolic blood pressure (SBP) ≥140 mm Hg or diastolic blood pressure (DBP) ≥95 mmHg; measured waist-to-hip ratio >1.0 for men and >0.8 for women.

When used herein, the term “multiple” means more than one.

When used herein, the terms “treatment,” “treat,” “treating,” and the like, are meant to include slowing or attenuating the progression of a disease or disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

When used herein in connection with the risk of a major adverse cardiovascular event, the terms “reduce,” “reduced,” “reduces,” “reducing,” and the like, refer to a reduction in the probability of the occurrence of a major adverse cardiovascular event. When used herein, the term “delaying the occurrence” of a major adverse cardiovascular event, means increasing the period of time until the occurrence of a major adverse cardiovascular event.

When used herein in connection with multiple outcomes, the term “composite” refers to the first to occur of any of the outcomes.

When used herein, the term “hazard ratio” refers to a measure of the relative rate of progression to an endpoint as compared to a control group. In outcome-based clinical trials, such as the CVOTs described herein, a reduction in the hazard ratio for a test arm as compared to the control indicates the therapy used in the test arm reduces the risk of the endpoint, in the case of the studies described herein, major adverse cardiovascular events.

The methods and uses described herein may be provided in simultaneous or sequential combination with a standard of care for reducing the risk of major adverse cardiovascular events, which includes administering the maximum tolerated dose of ACE inhibitors and ARBs, and adequate treatment of blood pressure, lipids, and HbA1c to the local guidelines. In certain embodiments, the methods described herein further comprise administering to the patient the maximum tolerated dose of an ACE inhibitor. In certain embodiments, the methods described herein further comprise administering to the patient the maximum tolerated dose of an ARB. Other agents which may be administered include beta blockers, calcium channel blockers, diuretics, antithrombotic agents, aspirin and statins.

“Therapeutically effective amount” means the amount of dulaglutide for the methods and uses of the present invention or pharmaceutical composition comprising dulaglutide for the methods and uses of the present invention that will elicit the biological or medical response of or desired therapeutic effect on the patient that is being sought by the researcher, medical doctor, or other clinician. An effective amount of dulaglutide may vary according to factors such as the disease state, age, sex, and weight of the individual, and the ability of dulaglutide to elicit a desired response in the individual. An effective amount is also one in which any toxic or detrimental effect is outweighed by the therapeutically beneficial effects. In certain embodiments, the therapeutically effective amount of dulaglutide for use in the methods described herein is selected from the group consisting of 1.5, 3.0 and 4.5 mg. In certain embodiments, the therapeutically effective amount of dulaglutide is 3.0 mg. In certain embodiments, the therapeutically effective amount of dulaglutide is 4.5 mg. In preferred embodiments, the therapeutically effective amount of dulaglutide is 1.5 mg.

Additional embodiments of the present invention are described below:

A method of reducing the risk of major adverse cardiovascular events in a patient with type 2 diabetes mellitus, comprising administering dulaglutide in a therapeutically effective amount to the patient once weekly, wherein the patient has type 2 diabetes mellitus and either: multiple cardiovascular risk factors without established cardiovascular disease; or established cardiovascular disease.

A method of reducing the risk of major adverse cardiovascular events in a patient with type 2 diabetes mellitus, comprising: identifying a patient having type 2 diabetes mellitus and either multiple cardiovascular risk factors without established cardiovascular disease or established cardiovascular disease; and administering dulaglutide in a therapeutically effective amount to the patient once weekly.

A method of delaying the occurrence of major adverse cardiovascular events in a patient with type 2 diabetes mellitus, comprising administering dulaglutide in a therapeutically effective amount to the patient once weekly, wherein the patient has type 2 diabetes mellitus and either: multiple cardiovascular risk factors without established cardiovascular disease; or established cardiovascular disease.

A method of improving glycemic control and reducing the risk of first occurrence of a major adverse cardiovascular event in a patient with type 2 diabetes mellitus, comprising administering dulaglutide in a therapeutically effective amount to the patient once weekly, wherein the patient has type 2 diabetes mellitus and either: multiple cardiovascular risk factors without established cardiovascular disease; or established cardiovascular disease.

A method of improving glycemic control in a patient with type 2 diabetes mellitus, comprising administering dulaglutide in a therapeutically effective amount to the patient once weekly, wherein the patient has type 2 diabetes mellitus and either: multiple cardiovascular risk factors without established cardiovascular disease; or established cardiovascular disease; and wherein the risk of a major adverse cardiovascular event in the patient is reduced.

In an embodiment, the risk of a major adverse cardiovascular event is reduced by at least about 10%.

In an embodiment, the risk of a major adverse cardiovascular event is reduced by at least about 11%.

In an embodiment, the risk of a major adverse cardiovascular event is reduced by about 12%.

In an embodiment, the risk of cardiovascular death is lower.

In an embodiment, the risk of non-fatal stroke is lower.

In an embodiment, the risk of non-fatal myocardial infarction is lower.

In an embodiment, the risk of the occurrence of a composite of the following outcomes is reduced: diabetic retinopathy needing laser, anti-VEGF therapy, or vitrectomy; clinical proteinuria; a 30% decline in eGFR; or chronic renal replacement therapy.

In an embodiment, the patient has multiple cardiovascular risk factors without established cardiovascular disease.

In an embodiment, the risk factors for cardiovascular disease are selected from the group consisting of: current tobacco use (any form of tobacco); use of at least 1 approved lipid modifying therapy to treat hypercholesterolemia or a documented untreated low-density lipoprotein cholesterol (LDL-C) ≥3.4 mmol/L (130 mg/dL) within the past 6 months; documented treated or untreated high-density lipoprotein cholesterol (HDL-C) <1.0 mmol/L (40 mg/dL) for men and <1.3 mmol/L (50 mg/dL) for women or triglycerides ≥2.3 mmol/L (200 mg/dL) within the past 6 months; use of at least 1 blood pressure medication to treat hypertension or untreated systolic blood pressure (SBP) ≥140 mm Hg or diastolic blood pressure (DBP) ≥95 mmHg; measured waist-to-hip ratio >1.0 for men and >0.8 for women.

In an embodiment, the amount of dulaglutide is selected from the group consisting of about 1.5 mg, about 3.0 mg and about 4.5 mg.

In an embodiment, the amount of dulaglutide is about 1.5 mg.

In an embodiment, the amount of dulaglutide is about 3.0 mg.

In an embodiment, the amount of dulaglutide is about 4.5 mg.

In an embodiment, once weekly administration of dulaglutide is continued for approximately 5 years.

In an embodiment, the patient is also administered the standard of care for reducing the risk of major adverse cardiovascular events.

In an embodiment, the patient is also administered the maximum tolerated dose of an ACE inhibitor.

In an embodiment, the patient is also administered the maximum tolerated dose of an ARB

In an embodiment, the patient is also administered a beta blocker.

In an embodiment, the patient is also administered a calcium channel blocker.

In an embodiment, the patient is also administered a diuretic.

In an embodiment, the patient is also administered an antithrombotic agent.

In an embodiment, the patient is al so administered aspirin.

In an embodiment, the patient is also administered a statin.

Dulaglutide for use in any of the above embodiments.

Use of dulaglutide in the preparation of a medicament for any of the above embodiments.

### EXAMPLES

A phase 3 clinical study named Researching Cardiovascular Events with a Weekly INcretin in Diabetes (REWIND) is designed to assess the effect of once-weekly administration of dulaglutide compared to placebo on major adverse CV events when added to the existing antihyperglycemic regimen of patients with type 2 diabetes who are at high risk for CV events. The enrollment criteria, set forth in Table 3 below, are designed to include participants who are similar to patients seen within a typical diabetes practice, who have varying cardiovascular risk factors or established cardiovascular disease:

TABLE 3
Enrollment Criteria.
Key inclusion criteria

T2DM with HbA1c ≤9.5%
Stable dose of 0, 1 or 2 oral glucose-lowering drugs ±
basal insulin for ≥3 months
BMI ≥23 kg/m²
If age ≥50 years, at least 1 of: prior MI; prior ischaemic
stroke; coronary revascularization ≥2 years earlier;
carotid or peripheral revascularization ≥2 months earlier;
unstable angina hospitalization; image proven myocardial
ischaemia; or percutaneous coronary intervention
If age ≥55 years, any of the above or at least 1 of:
documented myocardial ischaemia by stress test or imaging;
>50% coronary, carotid or lower extremity artery stenosis;
ankle-brachial index <0.9; eGFR persistently <60 mL/min/
1.73 m2; hypertension with left ventricular hypertrophy; or
persistent albuminuria
If age ≥60 years, any of the above or at least 2 of: any
tobacco use; use of lipid-modifying therapy or a documented
untreated LDL cholesterol ≥3.4 mmol/L (130 mg/dL) within
the past 6 months; HDL cholesterol <1.0 mmol/L (40 mg/dL)
for men and <1.3 mmol/L (50 mg/dL) for women or
triglycerides ≥2.3 mmol/L (200 mg/dL) within the past 6 months;
use of ≥1 blood pressure drug or untreated systolic blood
pressure ≥140 mm Hg or diastolic blood pressure ≥95 mm Hg;
or waist-to-hip ratio >1.0 (men) and >0.8 (women)
Run-in adherence to study drug = 100%
Signed informed consent

The study is designed to consist of a screening visit followed by a single-blind 3 week placebo run-in period. Afterwards, patients are randomized to either dulaglutide 1.5 mg or placebo and followed at approximately 6-month intervals. Patients are followed until approximately 1200 patients experience a primary endpoint event, adjudicated as such.

The primary efficacy measure is time to first occurrence (after randomization) of the composite endpoint of death from CV causes, nonfatal myocardial infarction (MI), or nonfatal stroke. Secondary outcomes include each component of the primary composite cardiovascular outcome, a composite clinical microvascular outcome comprising retinal or renal disease, hospitalization for unstable angina, heart failure requiring hospitalization or an urgent heart failure visit, and all-cause mortality. These outcomes are noted in Table 4. All deaths and cardiovascular, pancreatic and thyroid events (i.e. both efficacy and safety outcomes) are adjudicated by an external adjudication committee, which is blinded to treatment allocation.

TABLE 4
Secondary and safety outcomes.

| Secondary outcomes | Safety outcomes |
|---|---|
| Composite microvascular | Acute pancreatitis |
| outcome: diabetic retinopathy | |
| needing laser, anti-VEGF therapy, | |
| or | |
| vitrectomy; or clinical proteinuria; | |
| or a 30% decline in eGFR; or | |
| chronic renal replacement therapy | |
| Unstable angina hospitalization | Serious gastrointestinal events |
| Heart failure hospitalization or | Cancers: pancreatic, medullary |
| urgent visit | thyroid, other |
| | thyroid, other (excluding non- |
| | melanoma |
| | skin cancers) |
| Non-fatal MI | Severe hypoglycaemia |
| Non-fatal stroke | Immune reactions |
| Cardiovascular death | Serious hepatic events |
| Death | Serious renal events |
| | Supraventricular |
| | arrhythmias and cardiovascular |
| | conduction disorders |
| | Drug discontinuation |

Abbreviations:
VEGF, vascular endothelial growth factor;
eGFR, estimated glomerular filtration rate.

Sample size calculations are based on a 3-year recruitment period, an anticipated primary outcome event rate of 2% per year in the control group, annual dropout rate of 0.15%, and a 2-sided type I error of 5%. These assumptions indicate that recruitment of 9600 patients would result in a total of 1200 participants with at least 1 primary cardiovascular outcome over a maximum follow-up period of 8 years, and would provide 90% power to detect a hazard ratio of 0.82 for cardiovascular events. Follow-up ends after 1200 participants have had a primary cardiovascular outcome confirmed by adjudication.

All efficacy and safety analyses are designed to be conducted using an intention-to-treat approach that includes all randomized participants regardless of adherence. Baseline continuous variables are summarized as either means or medians with their standard deviations or interquartile ranges, and categorical variables are intended to be summarized as the number and percentage. The effect of the intervention on the time to the first occurrence of the primary outcome are designed to be analyzed using Cox proportional hazards models with the only independent variable being allocation to dulaglutide vs placebo. The proportional hazard assumptions are to be assessed graphically. Kaplan-Meier curves are to be generated along with log-rank P values. The incidence rates per 100 person years are to be calculated for each treatment group for all key outcomes. All secondary outcomes are to be analyzed in a predetermined order defined by a graphical approach to control the overall type I error. If the null hypothesis of no effect is rejected for the primary outcome, the graphical testing approach allocates the a parsimoniously for each secondary outcome. Exploratory subgroups to be examined include patients with prior CVD vs. those with no known CVD. For subgroup analyses, an interaction P value of <0.1 is considered suggestive of an interaction. No adjustments for multiplicity are to be performed.

12,137 individuals were screened, and 9901 individuals in 370 sites located in 24 countries were randomly allocated to either dulaglutide or placebo. The main reasons for not being randomized include not meeting eligibility criteria (68%) or personal decision (25%). The first participant was randomized in August 2011 and recruitment ended in August 2013. As noted in Tables 5 and 6, the mean age of participants was 66 years, the mean BMI was 32 kg/m 2 and 31% had a history of CVD (defined as a history of MI, ischaemic stroke, revascularization, hospitalization for unstable angina with concordant new ischaemic ECG changes, or a positive stress test with concordant imaging). In addition, 93% had a history of hypertension, 9% had a history of prior heart failure, and mean blood pressure was 137/78 mmHg. The mean reported duration of diabetes was 10 years, 24% of participants were taking insulin, 81% were taking metformin, 57% were on a sulphonylurea, and the mean baseline HbA1c was 7.3%. An angiotensin-converting enzyme (ACE) inhibitor or angiotensin receptor blocker (ARB) was used by 81% of participants, 45% were taking a β-blocker, 66% were taking a statin at baseline, 51% were on acetylsalicylic acid, 8% were on other antiplatelet agents, and the mean baseline LDL cholesterol was 2.56 nmol/L.

TABLE 5
Baseline clinical characteristics of 9901 randomized participants.

| Characteristic | All participants |
|---|---|
| Age, years: mean (s.d.) | 66.2 (6.5) |
| Females, n (%) | 4589 (46.3) |
| Geography, n (%) | |
| USA and Canada | 2071 (20.9) |
| Mexico and South America | 3021 (30.5) |
| Europe, Russia and South Africa | 4339 (43.8) |
| Asia: Taiwan and Korea | 148 (1.5) |
| Pacific: Australia and New Zealand | 322 (3.3) |
| Prior cardiovascular disease | 3111 (31.4) |
| (≥1 of the following 6), n (%) | |
| Prior MI | 1600 (16.2) |
| Prior ischemic stroke | 526 (5.3) |
| Prior unstable angina | 587 (5.9) |
| Prior revascularization^{a} | 1787 (18.1) |
| Prior hospitalization for ischaemia- | 1193 (12.1) |
| related events^{b} | |
| Prior documented | 922 (9.3) |
| myocardial ischaemia | |
| Prior hypertension, n (%) | 9223 (93.2) |
| Prior heart failure, n (%) | 852 (8.6) |
| Prior diabetic retinopathy, n (%) | 891 (9.0) |
| Prior fracture, n (%) | 1510 (15.3) |
| Prior cholecystectomy, n (%) | 1465 (14.8) |
| Current tobacco use, n (%) | 1407 (14.2) |
| Diabetes duration, years: mean (s.d.) | 10.0 (7.2) |
| Weight, kg: mean (s.d.) | 88.7 (18.5) |
| BMI, kg/m²: mean (s.d.) | 32.3 (5.7) |
| Blood pressure, mm Hg: mean (s.d.) | 137.2 (16.8)/78.5 (9.8) |
| Pulse, beats/min: mean (s.d.) | 71.5 (10.9) |
| Male waist-to-hip ratio: mean (s.d.) | 110.6 (13.1)/108.4 (11.2) |
| Female waist-to-hip ratio: mean (s.d.) | 106.7 (13.1)/113.3 (13.7) |
| HbA1c, %: mean (s.d.) | 7.3 (1.1) |
| Cholesterol, mmol/L: mean (s.d.) | 4.52 (1.16) |
| LDL cholesterol, mmol/L: mean (s.d.) | 2.56 (0.98) |
| HDL cholesterol, mmol/L: mean (s.d.) | 1.18 (0.34) |
| Triglycerides, mmol/L: median (IQR) | 1.60 (1.17, 2.22) |
| eGFR, mL/min/1.73 m2: mean (s.d.) | 77.6 (24.1) |
| eGFR <60 mL/min/1.73 m², n (%) | 2199 (22.2) |
| Albumin/creatinine, mg/mmol: | 1.94 (0.75, 8.02) |
| median (IQR) | |
| Macro or microalbuminuria^{c}, n (%) | 3491 (35.3) |

Abbreviations:
IQR, interquartile range;
s.d., standard deviation;
^{a}Coronary, carotid or peripheral;
^{b}Unstable angina or myocardial ischaemia on imaging, or need for percutaneous coronary intervention;
^{c}Albumin/creatinine ≥3.39 mg/mmol.

TABLE 6
Baseline use of drug classes in randomized participants.

| Diabetes-specific drugs classes | | Other drug classes | |
|---|---|---|---|
| None | 600 (6.1) | ACE inhibitor | 4909 (49.6) |
| Only 1 oral agent | 4926 (49.8) | ARB | 3366 (34.0) |
| Only 2 oral agents | 3894 (39.3) | ACE inhibitor | 8054 (81.4) |
| Any insulin | 2398 (24.2) | or ARB | |
| Metformin | 8016 (81.0) | Aldosterone | 464 (4.7) |
| Glibenclamide/ | 1271 (12.8) | antagonist | |
| glyburide | | All diuretic | 4592 (46.4) |
| Other sulfonylureas | 4373 (44.2) | Thiazides | 652 (6.6) |
| DPP-4 inhibitors | 88 (0.9) | β blocker | 4502 (45.5) |
| SGLT2 inhibitors | 12 (0.1) | Ca channel blocker | 3385 (34.2) |
| Meglitinides | 64 (0.7) | Acetylsalicylic acid | 5001 (50.5) |
| α-Glucosidase | 118 (1.2) | Other antiplatelet | 820 (8.3) |
| inhibitors | | Statin | 6537 (66.0) |
| Thiazolidinediones | 168 (1.7) | Fibrate | 892 (9.0) |
| Dopamine agonist | 47 (0.5) | Other lipid drug | 112 (1.1) |
| Other | 84 (0.9) | Proton pump inhibitor | 1673 (16.9) |

Values represent counts and percentage of all randomized.

Patients were and followed until August 2018. During a median follow-up of 5.4 years (interquartile range 5.1, 5.9) comprising 51,820 person-years, the final composite outcome status was known in 9610 patients. 1731 participants allocated to dulaglutide and 1761 participants allocated to placebo had at least 1 discontinuation of study drug during follow-up, while 4277 allocated to dulaglutide and 4196 allocated to placebo were taking study drug at the last visit. Participants allocated to dulaglutide or placebo respectively took study drug for 85.8% and 87.1% of the follow-up time from randomization until either they experienced the primary outcome or had a final follow-up.

Results are provided in Tables 7 and 8 below.

TABLE 7
Effect of dulaglutide on the primary and secondary outcomes.

| | Dulaglutide | Placebo | |
|---|---|---|---|
| | (N = 4949) | (N = 4952) | HR |
| Outcome | N (%) | N (%) | (95% CI) |
| MACE | 594 (12.0) | 663 (13.4) | 0.88 (0.79, 0.98) |
| MI | 223 (4.5) | 231 (4.7) | 0.96 (0.80, 1.15) |
| Stroke | 158 (3.2) | 205 (4.1) | 0.76 (0.62, 0.94) |
| CV Death | 317 (6.4) | 346 (7.0) | 0.91 (0.78, 1.06) |
| Composite | 1072 (21.7) | 1221 (24.7) | 0.85 (0.78, 0.92) |
| microvascular | | | |
| Unstable angina | 88 (1.8) | 77 (1.6) | 1.14 (0.84, 1.55) |
| Heart failure | 213 (4.3) | 226 (4.6) | 0.93 (0.77, 1.12) |
| All mortality | 536 (10.8) | 592 (12.0) | 0.90 (0.80, 1.01) |

As seen in Table 7, a weekly injection of dulaglutide significantly and safely reduced the hazard of CV outcomes by 12% compared to placebo. Moreover, the benefit was consistent across all 3 components of the composite primary outcome, with the largest estimated effect size being noted for nonfatal stroke. The incidence of the composite microvascular outcome was also lower in participants allocated to dulaglutide versus placebo.

TABLE 8
Subgroup analysis.

| | Dulaglutide | Placebo | | |
|---|---|---|---|---|
| | Events/ | Events/ | HR | P value for |
| Subgroup | Total (%) | Total (%) | (95% CI) | Interaction |
| Prior CVD | 280/1560 | 315/1554 | 0.87 | 0.80 |
| | (17.9) | (20.3) | (0.74-1.02) | |
| No Known | 314/3389 | 3487/3398 | 0.89 | |
| Prior CVD | (9.3) | (10.2) | (0.76-1.04) | |

As seen in table 8, the positive effect of dulaglutide on the primary outcome was similar in participants with and without a prior CV event.

1. A method of reducing the risk of major adverse cardiovascular events in a patient with type 2 diabetes mellitus, comprising administering dulaglutide in a therapeutically effective amount to the patient once weekly, wherein the patient has type 2 diabetes mellitus and either:
(a) multiple cardiovascular risk factors without established cardiovascular disease; or
(b) established cardiovascular disease.

2. A method of reducing the risk of major adverse cardiovascular events in a patient with type 2 diabetes mellitus, comprising:
(a) identifying a patient having type 2 diabetes mellitus and either:
i) multiple cardiovascular risk factors without established cardiovascular disease; or
ii) established cardiovascular disease; and
(b) administering dulaglutide in a therapeutically effective amount to the patient once weekly.

3. A method of improving glycemic control and reducing the risk of first occurrence of a major adverse cardiovascular event in a patient with type 2 diabetes mellitus, comprising administering dulaglutide in a therapeutically effective amount to the patient once weekly, wherein the patient has type 2 diabetes mellitus and either:
(a) multiple cardiovascular risk factors without established cardiovascular disease; or
(b) established cardiovascular disease.

4. The method of claim 1 wherein the patient has multiple cardiovascular risk factors without established cardiovascular disease.

5. The method of claim 4, wherein the cardiovascular risk factors are selected from the group consisting of (a), (b), (c), (d) and (e):
(a) tobacco use;
(b) at least 1 of:
i) use of at least 1 approved lipid modifying therapy to treat hypercholesterolemia; or
ii) a documented untreated low-density lipoprotein cholesterol (LDL-C)≥3.4 mmol/L (130 mg/dL) within the past 6 months;
(c) at least 1 of:
i) high-density lipoprotein cholesterol (HDL-C) measurement within the past 6 months of: <1.0 mmol/L (40 mg/dL) for men; and <1.3 mmol/L (50 mg/dL) for women; or
ii) triglycerides ≥2.3 mmol/L (200 mg/dL) within the past 6 months;
(d) at least 1 of:
i) use of at least 1 blood pressure medication to treat hypertension; or
ii) untreated systolic blood pressure (SBP)≥140 mm Hg or diastolic blood pressure (DBP)≥95 mmHg; and
(e) measured waist-to-hip ratio >1.0 for men and >0.8 for women.

6. The method of any of claim 1 wherein the patient has established cardiovascular disease.

7. The method of claim 1 wherein the method reduces the risk of a major adverse cardiovascular event by at least about 10%.

8. The method of claim 1 wherein the therapeutically effective amount of dulaglutide is selected from the group consisting of 1.5 mg, 3.0 mg and 4.5 mg.

9. The method of claim 1 wherein the therapeutically effective amount of dulaglutide is 1.5 mg.

10. The method of claim 1 wherein the once weekly administration of dulaglutide is continued for approximately 5 years.

11. The method of claim 1 further comprising administering to the patient one or more of the following: an angiotensin converting enzyme (ACE) inhibitor; an angiotensin receptor blocker (ARB); a beta blocker; a calcium channel blocker; a diuretic; an antithrombotic agent; aspirin or a statin.


SEQUENCE LISTING (1 sequence)

SEQ ID NO: 1 - protein, 275 aa, synthetic construct
  REGION 1..275: note = synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG